(19) United States
(12) Patent Application Publication
(10) Pub. No.: US 2018/0333107 A1
(43) Pub. Date: Nov. 22, 2018

(54) NON-INVASIVE WEARABLE DEVICE, PROCESS AND SYSTEMS WITH ADJUSTABLE OPERATION

(71) Applicant: Rocket Business Ventures, S.A. de C.V., San Pedro Garza Garcia (MX)

(72) Inventors: Fernando García Sada, San Pedro Garza Garcia (MX); Eduardo García Alanís, San Pedro Garza Garcia (MX); Sergio de Jesús Elizondo Muñoz, San Pedro Garza Garcia (MX); Daniel David Pechir Rivera, San Pedro Garza Garcia (MX); Patricia Alejandra Vázquez Marines, San Pedro Garza Garcia (MX); Maria Catalina Charles Herrera, San Pedro Garza Garcia (MX)

(73) Assignee: Rocket Business Ventures, S.A. de C.V., San Pedro Garza Garcia (MX)

(21) Appl. No.: 15/981,670

(22) Filed: May 16, 2018

Related U.S. Application Data
(60) Provisional application No. 62/506,680, filed on May 16, 2017.

(51) Int. Cl. A61B 5/00; A61B 5/0476; A61B 5/0496; A61B 5/0488; A61B 5/0402; A61B 5/01; A61B 5/1477; A61B 5/1486; A61B 5/05; A61B 5/145; A61B 5/053; A61B 5/026; A61B 5/11

(52) U.S. Cl. CPC A61B 5/7405; A61B 2576/02; A61B 5/0476; A61B 5/0496; A61B 5/0488; A61B 5/0402; A61B 5/0073; A61B 5/0075; A61B 5/0095; A61B 5/01; A61B 5/0033; A61B 5/1477; A61B 5/1486; A61B 5/0022; A61B 5/742; A61B 5/7455; A61B 5/7275; A61B 5/05; A61B 5/14535; A61B 5/14532; A61B 5/14546; A61B 5/0537; A61B 5/026; A61B 5/1116; A61B 2562/0204; A61B 2562/0219; A61B 2562/0238; A61B 2562/0271; A61B 2562/063; A61B 2562/164; A61B 5/6802

(57) ABSTRACT

A wearable device for attachment to a body part of a user is for sensing, feedback and adjusting features. The wearable device includes a flexible housing, an array of sensing devices configured to be positioned proximate the body part to determine respective physiological characteristics of the user, and a processor configured to receive and process information regarding the physiological characteristics from the array of sensing devices. A communication module is associated with the processor and configured to communicate data to/from an external control unit (ECU). The processor is configured to receive adjustment signals from the ECU and adjust the sensing devices for performance control according to an image registration process that includes mapping a position and arrangement of the sensing devices relative to anatomical structures defined in an anatomical model to generate a mapped anatomical model, and generating tissue-related measurements within the mapped anatomical model based upon targeted anatomical structures within a sensitivity path relative to the sensing devices.

### CROSS-REFERENCE TO RELATED APPLICATIONS

This application claims priority from U.S. Provisional Patent Application Ser. No. 62/506,680 filed May 16, 2017, titled “NON-INVASIVE WEARABLE DEVICE, SOFTWARE AND SYSTEM TO IMPROVE HEALTH AND WELLNESS”, (Attorney Docket No. 3061.00002), Assigned to Rocket Business Ventures, S.A. de C.V., the contents of which are incorporated by reference in its entirety except to the extent disclosure therein is inconsistent with disclosure herein.

### FIELD OF INVENTION

The field of invention relates in general to computational science and biomedical sensors, and, more particularly, to wearable devices and the provision of health-related notifications to users.

### BACKGROUND

This background information is provided to reveal information believed by the applicant to be of possible relevance to the present invention. No admission is necessarily intended, nor should be construed, that any of the preceding information constitutes prior art against the present invention.

According to the World Health Organization (WHO, 2015), around 70% of the deaths worldwide are caused by non-communicable diseases (NCDs) (e.g. see http://www.who.int/mediacentre/factsheets/fs355/en). The increase in noncommunicable diseases, such as heart diseases, stroke, cancer, diabetes and chronic lung cancer, has been attributed to major risk factors related to inadequate lifestyle habits. The preventable condition of NCDs motivates the development of technology able to potentiate the integration and preservation of behavioral patterns that contribute to improve and maintain personal state of health and wellness.

Today, a wide variety of wearable devices are able to continuously provide non-invasive measures of the state of health and level of activity of individuals. The aforementioned measures are then processed to provide notifications to inform the results and/or suggest further action.

Advances in biomedical sensing, data analytics and communications have been combined to provide biomedical measurements within expanded notification systems and sets of functionalities that seek to enhance health and wellness. Due to the broad advantages of wearable technology, continuous research and development efforts are directed to expanding the applicability of wearable technology for clinical purposes, facing challenges such as enhancing the accuracy of the measurements provided by these devices while improving energy expenditure and safety effects associated with the continuous and direct interaction with the users in their daily-life routine.

A need for adjustability of the wearable device and associated sensors may exist to improve the quality and accuracy of the assessment, feedback and notifications.

### BRIEF SUMMARY OF THE INVENTION

The present embodiments offer a set of approaches to the above-mentioned challenges, providing a wearable device that is able to provide health-related notifications to the user and may forecast possible health conditions including adjusting performance using reference data targeted to the user.

Embodiments are directed to wearable device configured for attachment to a body part of a user for sensing, feedback and adjusting features. The wearable device includes a flexible housing configured to be attached to the body part of the user, an array of sensing devices carried by the housing and configured to be positioned proximate the body part to determine respective physiological characteristics of the user, and a processor configured to receive and process information regarding the physiological characteristics from the array of sensing devices. A memory is associated with the processor and configured to store the processed information, a communication module is associated with the processor and configured to communicate data to/from an external control unit (ECU), and at least one power source is configured to provide power to the array of sensing devices and the processor. The processor is configured to receive adjustment signals from the ECU and adjust the sensing devices for performance control according to an image registration process that includes mapping a position and arrangement of the sensing devices relative to anatomical structures defined in an anatomical model to generate a mapped anatomical model, and generating tissue-related measurements within the mapped anatomical model based upon targeted anatomical structures within a sensitivity path relative to the sensing devices.

Additionally, and/or alternatively, the flexible housing comprises an elastic band, a breathable, stretchable, conformable band, and/or a shape-memory polymer band. Also, the flexible housing may further include a compression mechanism controlled by the processor to adjust the fit and position of the sensing devices.

Additionally, and/or alternatively, the array of sensing devices is configured to determine physiological characteristics using at least one of an electroencephalogram (EEG), electrooculogram (EOG), electromiogram (EMG), electrocardiogram, (ECG), optical spectroscopy and tomography, optical photoacoustic spectroscopy and tomography, thermoacoustic spectroscopy and tomography, bioimpedance, bioimpedance tomography, acoustic sensing, enzyme-based and enzyme-free electrochemical sensing, temperature sensing, inertial mass unit measurements, and combinations thereof.

Additionally, and/or alternatively, the array of sensing devices comprises at least one of optical sensor arrays, acoustic sensor arrays, photoacoustic sensor arrays, bioimpedance sensor arrays, electrochemical biosensors, and inertial measurement units (IMU). The array of sensing devices may also include at least one of lasers, solid state light sources, light emitting diodes (LEDs), electro-acoustical transmitters, piezoelectric transducers, voltage electrodes, current electrodes, alternating voltage generators, voltage-to-current converters, colorimeters, miniaturized spectrometer systems, miniaturized charge-coupled devices (CCDs), photodiodes, avalanche photo-diodes (APD), phototransistors, photo-multiplying tubes, hybrid photodetector systems, optical fiber-based sensor systems, and micro resonator systems.

Additionally, and/or alternatively, the array of sensing devices are arranged in one or more rows within the flexible housing and include at least one of point measurements and tomographic measurements to provide at least one of transverse and axial image information as the physiological characteristics of the user.

Additionally, and/or alternatively, the array of sensing devices comprise reprogrammable materials configured to controllably change at least one of mechanical, electrical, optical and magnetic properties in response to the adjustment signals that include at least one of electronic signals, magnetic signals, and electromagnetic signals.

Additionally, and/or alternatively, the communication module associated with the processor and configured to communicate data to/from the ECU comprises at least one of a wireless communication module and a wired communication module.

Additionally, and/or alternatively, a feedback module is associated with the processor and configured to provide health-related notifications to the user using at least one of audible, visual and haptic notifications.

Embodiments of the present invention are also directed to a method for determining and providing health-related notifications to a user. The method includes: providing a wearable device, including an array of sensing devices, to be positioned proximate to a body part of the user; retrieving and processing anatomical information about the body part; generating an anatomical model of the body part based upon the anatomical information; and generating and communicating adjustment signals for the sensing devices based upon an image registration process that includes mapping a position and arrangement of the sensing devices relative to anatomical structures defined in the anatomical model to generate a mapped anatomical model, and generating tissue-related measurements within the mapped anatomical model based upon targeted anatomical structures within a sensitivity path relative to the sensing devices. The method further includes: determining local energy-related values within the anatomical model; obtaining measurements from the sensing devices and comparing the measurements with the local energy-related values to determine tomographic data of tissue properties regarding the body part; determining physiological parameters of the user based upon the tomographic data; analyzing the physiological parameters over a time period to obtain health-related metrics; and identifying correlations and trends based upon the health-related metrics to determine and provide the health-related notifications to the user.

Additionally, and/or alternatively, the wearable device includes an elastic band carrying the array of sensing devices to be positioned adjacent the skin of the body part. The elastic band may further comprise a compression mechanism controlled to adjust the fit and position of the sensing devices.

Additionally, and/or alternatively, obtaining measurements from the sensing devices includes using at least one of an electroencephalogram (EEG), electrooculogram (EOG), electromiogram (EMG), electrocardiogram, (ECG), optical spectroscopy and tomography, optical photoacoustic spectroscopy and tomography, thermoacoustic spectroscopy and tomography, bioimpedance, bioimpedance tomography, acoustic sensing, enzyme-based and enzyme-free electrochemical sensing, temperature sensing, inertial mass unit measurements, and combinations thereof.

Additionally, and/or alternatively, the array of sensing devices comprises at least one of optical sensor arrays, acoustic sensor arrays, photoacoustic sensor arrays, bioimpedance sensor arrays, electrochemical biosensors, inertial measurement units (IMU), lasers, solid state light sources, light emitting diodes (LEDs), electro-acoustical transmitters, piezoelectric transducers, voltage electrodes, current electrodes, alternating voltage generators, voltage-to-current converters, colorimeters, miniaturized spectrometer systems, miniaturized charge-coupled devices (CCDs), photodiodes, avalanche photo-diodes (APD), phototransistors, photo-multiplying tubes, hybrid photodetector systems, optical fiber-based sensor systems, and micro resonator systems.

Additionally, and/or alternatively, the array of sensing devices are arranged in one or more rows within the wearable device and include at least one of point measurements and tomographic measurements to provide at least one of transverse and axial image information as the tomographic data of the user.

Additionally, and/or alternatively, the array of sensing devices comprise reprogrammable materials; and the method further comprises controllably changing at least one of mechanical, electrical, optical and magnetic properties of the sensing device in response to the adjustment signals that include at least one of electronic signals, magnetic signals, and electromagnetic signals.

Additionally, and/or alternatively, retrieving and processing anatomical information about the body part includes communicating data from the wearable device to an external control unit using at least one of a wireless communication module and a wired communication module of the wearable device.

Additionally, and/or alternatively, retrieving and processing anatomical information about the body part also includes accessing stored reference anatomical information about the body part including at least one of previously determined physiological parameters of the user and general physiological parameters of the body part.

Additionally, and/or alternatively, determining local energy-related values within the anatomical model comprises determining at least one of optical properties, acoustic properties, and electrical properties for different tissue structures within the anatomical model using operational parameters of the sensing devices including at least one of wavelength, frequency, intensity, amplitude, and modulation.

Additionally, and/or alternatively, analyzing the physiological parameters to obtain health-related metrics includes obtaining at least one of deep-temperature distributions, concentration changes of hemoglobin and deoxy-hemoglobin, water, glucose, cholesterol, conductivity values, vitamin levels, cell-count, flow readings, and genetic predisposition markers.

Additionally, and/or alternatively, obtaining health-related metrics further includes obtaining at least one of hydration levels, caloric intake, cardiac frequency, stress-related measures, posture, voice pitch, equilibrium and balance, activity levels, and type of activity performed of the user.

Additionally, and/or alternatively, identifying correlations and trends based upon the health-related metrics to determine and provide notifications to the user further includes identifying cross-correlations and trends between health-related metrics.

Additionally, and/or alternatively, providing the health-related notifications to the user includes using at least one of audible, visual, haptic, and electronic notifications.

### BRIEF DESCRIPTION OF DRAWINGS

FIG. 1 is a schematic diagram depicting an arrangement of the different arrays of sensing devices in accordance with an embodiment of the present invention. Different sensing devices are arranged in rows within the wearable device.

FIG. 2A is a cross-sectional view of an anatomical model mapped on the human wrist anatomy of a user including relative positions of sensing devices along the wearable device with respect to the anatomical model in accordance with an embodiment of the present invention.

FIG. 2B a cross-sectional area of a human wrist illustrating an example of the array of sensing devices located in the wearable device and a pressure control mechanism to maintain stable contact between the skin and the sensing devices in accordance with an embodiment of the present invention.

FIG. 2C is a perspective view illustrating the different arrays of sensing devices (e.g. optical sensing devices, photoacoustic sensing devices) organized in rows with a tomographic configuration, including an exploded view showing a compartment including a sensing device with a pressure control mechanism that maintains stable contact between the skin and the sensing devices in accordance with an embodiment of the present invention.

FIG. 3 is a chart illustrating an example of the continuous recovery of the distribution of diverse tissue properties in accordance with an embodiment of the present invention.

FIG. 4 is a schematic diagram illustrating an example of the circuitry in an embodiment of the wearable device including sensing devices held in contact with the skin of the body part, and also an associated processor along with blocks such as memory, frequency generator, a feedback module and a communication module in accordance with an embodiment of the present invention.

FIG. 5 is a schematic diagram illustrating the communication process between the wearable device of FIG. 4, an external control unit (ECU) and a computer network in accordance with an embodiment of the present invention.

FIG. 6 is a schematic diagram illustrating a further example of the circuitry of FIG. 4) in an embodiment of the wearable device including an additional row of sensing devices (e.g. bioimpedance sensing devices) in accordance with an embodiment of the present invention.

FIG. 7 is a schematic diagram illustrating an example of the circuitry in an embodiment of the wearable device including the relative position of a pressure control mechanism with respect to the sensing devices within the wearable device in accordance with an embodiment of the present invention.

FIG. 8 is a flow-diagram illustrating a process for imaging an array of sensing devices with respect to the spatial distribution of the tissue properties and providing health-related notifications to a user in accordance with features of an embodiment of the invention.

FIGS. 9A-9D illustrate a flowchart of an exemplary process for generating adjusting signals for adjustment of the sensing devices and providing health-related notifications in accordance with an embodiment of the present invention.

FIGS. 10A and 10B are a flow chart illustrating a physical activity recognition process including calibration to generate data for a training process, resulting in a classification model to estimate future physical activity of the user in accordance with an embodiment of the present invention.

FIG. 11 is a flow chart illustrating a process to analyze physiological parameters over a specified time period to determine health-related metrics and corresponding notifications in accordance with an embodiment of the present invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

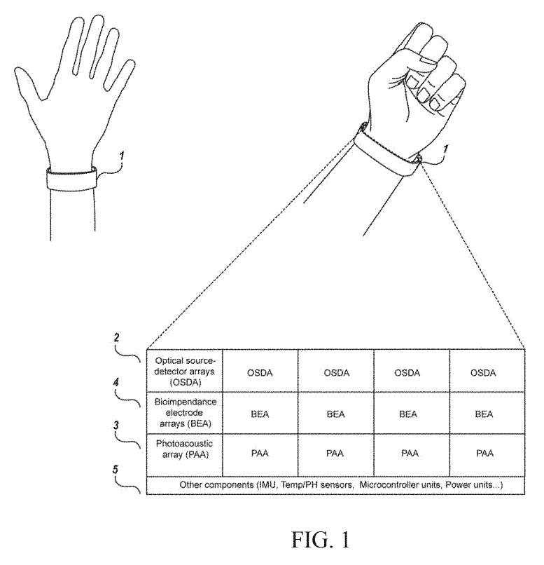

The embodiments disclosed relate to a wearable device 1 that provides noninvasive bodily measures and a method that delivers adjustment signals to sensing devices 2-5 for improved or optimal operation and feedback to a user. Referring initially to FIGS. 1-7, embodiments of the wearable device 1 are described.

FIG. 1 is a schematic diagram depicting an arrangement of the different arrays of sensing devices 2-5 in accordance with an embodiment of the present invention. Different sensing devices 2-5 are arranged in rows within the wearable device 1. Rows of sensing devices of the same type such as optical sensing devices 2, photoacoustic sensing devices 3, etc., are arranged in tomographic configuration while one or more rows of sensing devices may hold non-tomographic configurations (i.e. for pointwise measurements).

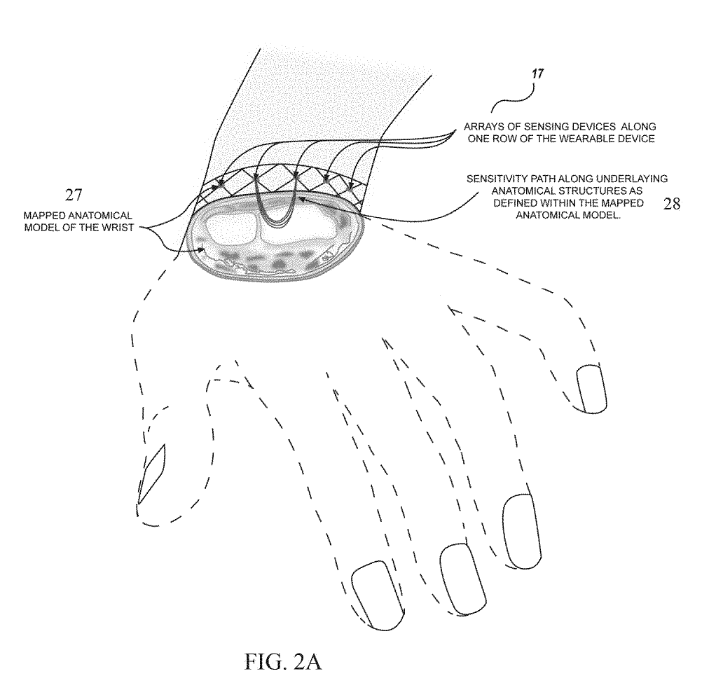

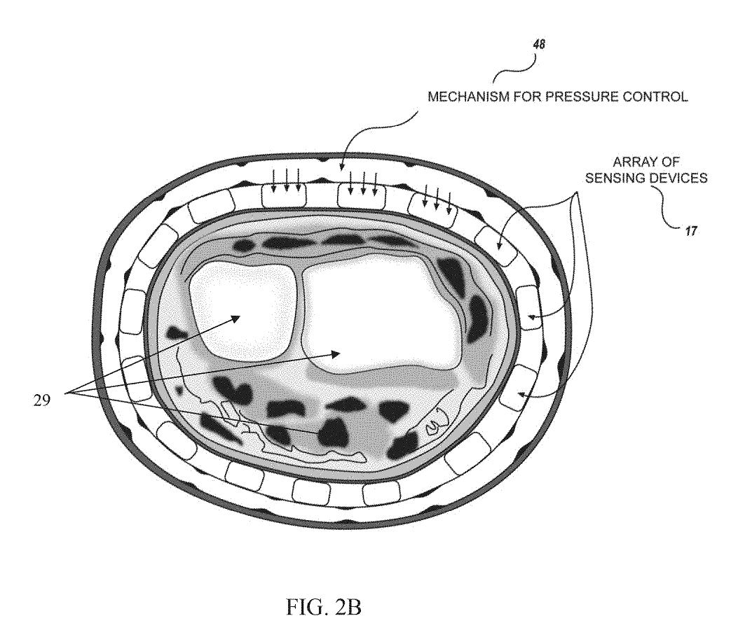

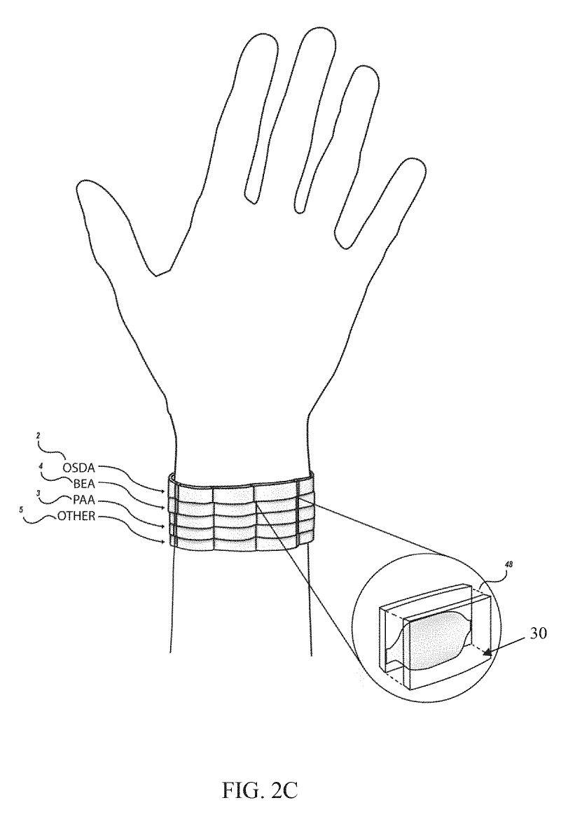

FIG. 2A is a cross-sectional view of an anatomical model 27 mapped on the human wrist anatomy of a user including relative positions of arrays 17 of sensing devices along the wearable device 1 and within a sensitivity path 28 with respect to the anatomical model 27 in accordance with an embodiment of the present invention. FIG. 2B a cross-sectional area of a human wrist illustrating an example of the array 17 of sensing devices located in the wearable device 1 and a pressure control mechanism 48 to maintain stable contact between the skin and the sensing devices in accordance with an embodiment of the present invention. FIG. 2C is a perspective view illustrating the different arrays of sensing devices 2-5 (e.g. optical sensing devices, photoacoustic sensing devices) organized in rows with a tomographic configuration, including an exploded view showing a cell or compartment 30 including a sensing device with a pressure control mechanism 48 that maintains stable contact between the skin and the sensing devices in accordance with an embodiment of the present invention. The tomographic configuration may account for the imaging capabilities of the wearable device 1. A group of different sensing devices 2-5 may be organized within one or more additional rows, providing pointwise measurements (e.g. electrochemical sensing devices, inertial measurement units, etc.).

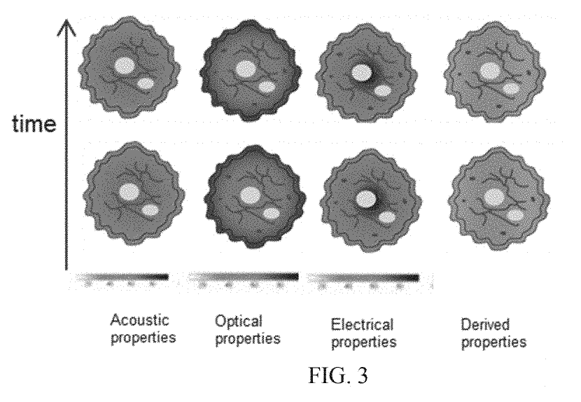

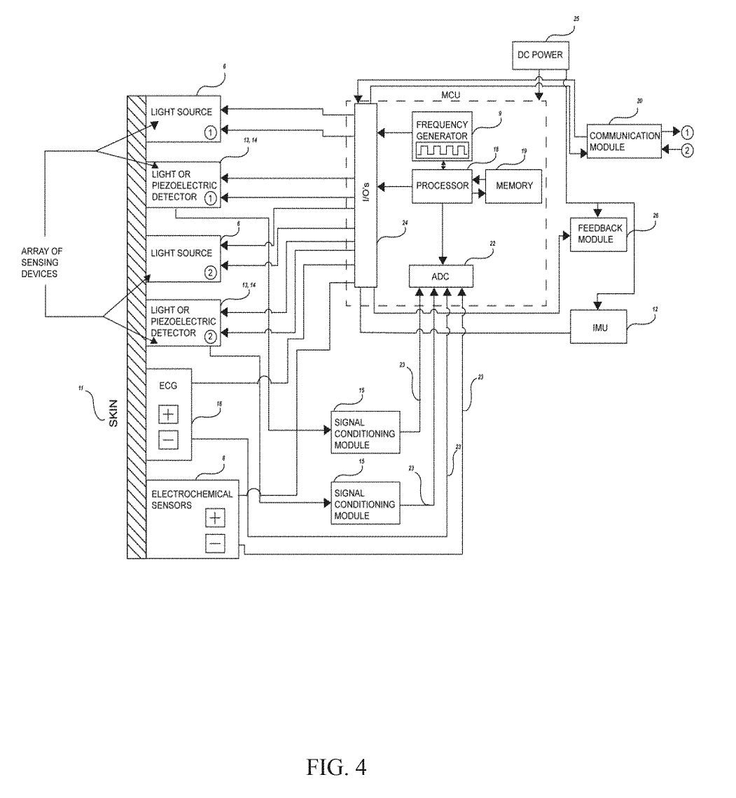

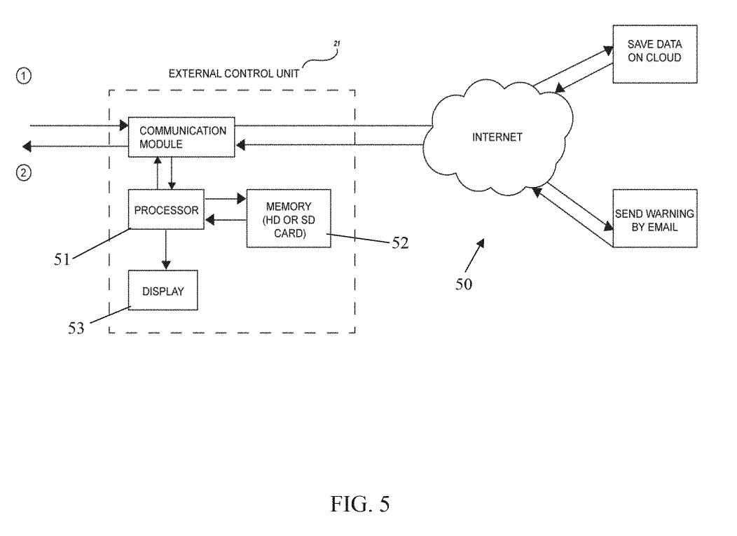

FIG. 3 is a chart illustrating an example of the continuous recovery of the distribution of diverse tissue properties in accordance with an embodiment of the present invention. FIG. 4 is a schematic diagram illustrating an example of the circuitry in an embodiment of the wearable device 1 including sensing devices held in contact with the skin of the body part, and also an associated processor 18 along with blocks such as memory 19, frequency generator 9, a feedback module 26 and a communication module 20 in accordance with an embodiment of the present invention. FIG. 5 is a schematic diagram illustrating the communication process between the wearable device 1 of FIG. 4, an external control unit (ECU) 21 and a computer network 50 in accordance with an embodiment of the present invention.

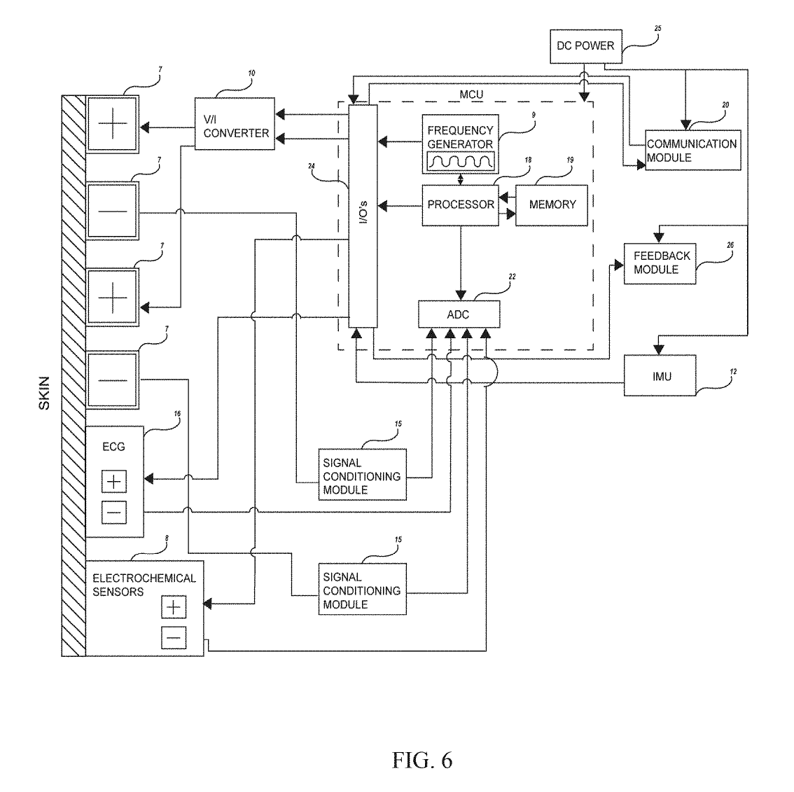

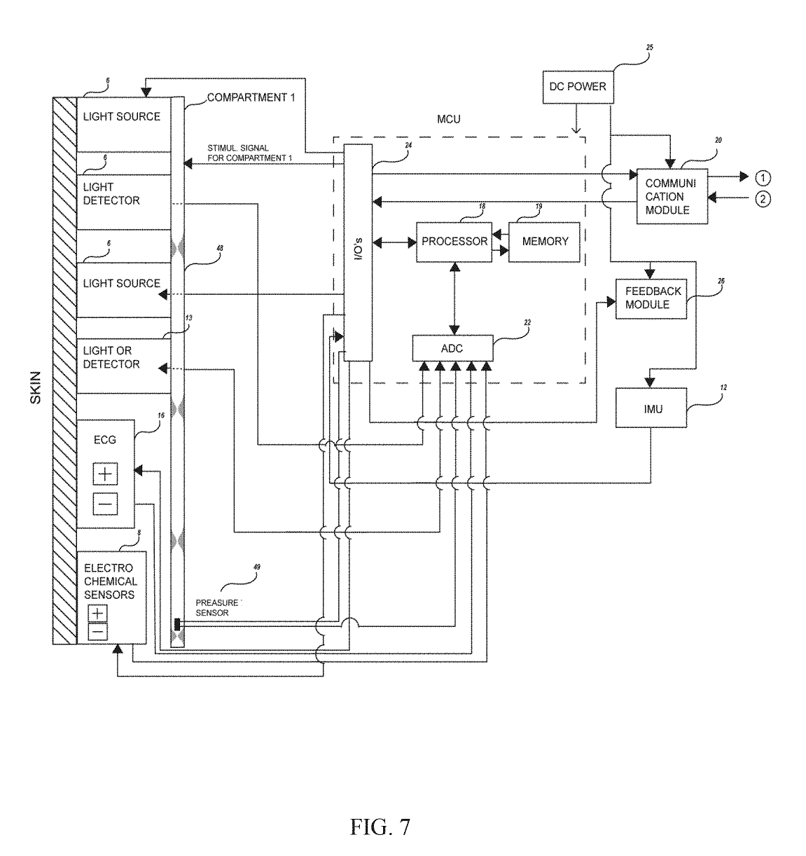

FIG. 6 is a schematic diagram illustrating a further example of the circuitry of FIG. 4) in an embodiment of the wearable device 1 including an additional row of sensing devices 7 (e.g. bioimpedance sensing devices) in accordance with an embodiment of the present invention. FIG. 7 is a schematic diagram illustrating an example of the circuitry in an embodiment of the wearable device 1 including the relative position of a pressure control mechanism 48 with respect to the sensing devices within the wearable device in accordance with an embodiment of the present invention. A pressure sensing device 49, or other type of sensing device, may be located within each cell or compartment 30 associated with each sensing device as part of the pressure control mechanism 48.

The wearable device 1 may comprise a flexible housing, which may be a band, a strap or any other design for covering the contour of a body part. The band may be a breathable, stretchable, conformable band, and/or a shape-memory polymer band. The wearable device can be attached to one or more body parts simultaneously, including the human wrists, ankles, forehead, etc., for example. The device may be attached to a user's wrist and it may include arrays of sensing devices 2-5 configured to be positioned proximate to the body part. The device may also include compartments for noninvasive body fluid sampling and analysis, such as urine, saliva, tears, etc.

Measurements provided by the wearable device include but are not limited to Electroencephalogram (EEG), Electrooculogram (EOG), Electromiogram (EMG), Electrocardiogram (ECG), Optical Spectroscopy and Tomography, Optical Photoacoustic spectroscopy and Tomography, Thermoacoustic Spectroscopy and Tomography, Bioimpedance, Bioimpedance Tomography, Acoustic sensing, Enzyme-based and enzyme-free electrochemical sensing, Temperature sensing, Inertial measurements, and combinations thereof.

Within this description, a sensing device may refer to various sensors including combinations of an energy source 6 and energy detector 13, 14 included in an array (e.g., as shown in FIG. 4). The arrays of sensing devices may include optical sensor arrays 2, photoacoustic sensor arrays 3 (or other acoustic sensor arrays), and bioimpedance sensor arrays 4, for example. Other sensing devices 5 may be included in the device such as electrochemical biosensors, inertial measurement unit (IMU), temperature sensors, PH sensors, etc. Other components such as a processor 18, memory 19, power unit 25 and other related circuitry may also be included as discussed further below.

In some examples, optical excitation 6 may employ lasers or other solid state light sources such as Light Emitting Diodes (LEDs), acoustic excitation may employ electro-acoustical transmitters such as piezoelectric transducers, etc., bioelectricity excitation 7 may employ electrodes to deliver voltage or current stimuli through the measurement site, electrochemical transduction 8 may employ alternating voltage generators 9 and voltage-to-current (V/I) converters 10 to deliver current signals across the measurement site 11, while chemical methods may employ chemical compounds to enable colorimetric techniques and an inertial measurement unit (IMU) 12 may provide inertial data.

Probe-hosted photodetection 13 may be achieved using miniaturized spectrometer systems, miniaturized Charge-Coupled Devices (CCDs), photodiodes, Avalanche Photo-diodes (APD), phototransistors, photo-multiplying tubes or hybrid photodetector systems. Detection of acoustic waves 14 may be achieved using sensors or arrays including piezoelectric transducers, optical fiber-based sensor systems, customized tuning forks and micro-resonator systems, etc. Bioimpedance detection may include inverting amplifiers and demodulators within one or more signal conditioning modules 15. Electrochemical sensing 8 and ECG sensing 16 may employ electrodes in different configurations.

Energy sources 6, 7, and energy detectors 13, 14 may be configured to deliver optical, mechanical and electric signals across the measurement site, while chemical methods may employ chemical compounds to enable colorimetric techniques. The IMU 12 may be configured to determine instant linear acceleration, angular velocity of a user along with on-site earth magnetic field measurements.

The arrays 17 of sensing devices may be arranged in reflectance configuration in order to collect the back-scattered energy waves exiting the tissue, as shown in FIGS. 2A and 2B. Both point measurements and tomographic measurements are considered in an exemplary embodiment of the invention. Tomographic arrangements of the arrays 17 of sensing devices may be considered to account for the imaging capabilities of the system. Arrays 17 of sensing devices may incorporate one or more rows of sensing devices 2-5 in order to provide both transverse and axial image reconstructions as shown in FIGS. 2A-2C. Arrays 17 of sensing devices may consider different spacing between the sensing devices. A group of different sensing devices may be organized within one or more additional rows 5, providing pointwise measurements (e.g. electrochemical sensing devices, inertial measurement unit).

Calibration surfaces may be present on one or more locations in the embodiments to obtain the reference measurements to compute absolute/relative quantities, or other necessary information relevant to the accuracy of the techniques, and also to evoke necessary adjustments in operational parameters. Examples of calibration surfaces may include 100% reflectance surfaces, total optical absorption materials, tissue-simulating phantoms with known acoustic properties and distributions, etc. The aforementioned processes may be performed during convenient conditions determined by the device, e.g. data for background subtraction routines may be captured at nighttime or low-light conditions. Also, in order to assess detectability of specific metabolites, absorbers, scatterers, etc. and derive the desired measures, quality assurance routines may be performed with the aforementioned calibration phantom materials.

The wearable device 1 may include a processor 18 and a memory 19 associated to the processor. A communication module 20 may be also associated to the processor in order to communicate with an External control unit (ECU) 21. The input analog signals are digitized within an analog-to-digital converter (ADC) 22 module and read by the processor 18. The processor directs the memory of data and instructions and executes the operations given by the instructions explained in further detail with respect to the method, below. The executed instructions may include digital filtering of the signals, detection of features within the signals and implementation of model-based or model-free algorithms and routines.

A frequency generator module 9 may be included for generating alternating output signals in order to modulate the energy sources and detectors. A V/I converter 10 is employed to convert the voltage supplied by the frequency generator 9 into a differential sinusoidal current. In this example, a control bus 23 associated with the processor 18 interfaces electrically with the ADC 22, memory 19, frequency generator module 9, etc. by transmitting continually updated adjustment signals derived from the execution of instructions associated with the method. A signal conditioning module 15 may be included to process the signals measured by the detectors in order to transmit processed signals into the processor 18. The input/output ports 24 associated with the processor 18 are interfaced electrically with sensing devices and associated devices (i.e. ADC, frequency generator, V/I converters, signal conditioning module, feedback module) in order to communicate continuously updated adjustment signals associated to the operation of the sensing devices.

The IMU 12 may be used to identify the type of physical activity that the user is performing at a certain time. By combining the data of linear acceleration, angular velocity and earth magnetic field given by a triaxial accelerometer, gyroscope and magnetometer, for example, the inertial data may be classified in order to decide which activity is performed by the user. A power source 25, such as a rechargeable battery, may be configured to provide power to the processor, sensing devices and associated devices.

The sensing devices 2-5 may be composed of reprogrammable materials that may reversibly change their mechanical, electrical, optical and magnetic properties in response to the adjustment signals delivered by the processor. Adjustment signals sent by the processor may further comprise different signals including electrical signals, magnetic signals, electromagnetic signals, mechanical signals, etc. Tunability of physical and chemical properties of the sensing devices 2-5 may be achieved by employing polymeric materials within the field of organic electronics, graphene-based electronic devices, and/or other configurable materials. The flexible housing of the wearable device 1 may be synthesized with polymeric substrates exhibiting breathability, stretchability and conformability with the skin such as those composed of shape-memory polymer substrates. Heat dissipation may be enhanced by employing directionality properties for heat conduction exhibited by materials such as graphene, germanene, etc.

Once the processor 18 has received and processed the information from the sensing devices and associated devices, it may send this data to a communication module 20 which then may transfer data to/from the ECU 21. The communication may be established using wired or wireless connections. Wireless connections may be stablished via Bluetooth or Wi-Fi, for example via the protocols IEEE 802.15.1 WPAN and IEEE 802.11 WLAN, for example. The ECU 21, such as a personal computer or a smartphone, for example, may have the capacity to process (e.g., via processor 51) the received data sent by the wearable device 1, saving the resultant analysis (e.g., via memory 52) and providing a summary of the health-related notifications from the user via a display device 53. At the same time, the ECU 21 may store the resultant analysis data on a cloud framework where the user may have previously assigned access, as illustrated in FIG. 5. The ECU 21 may be capable of sending email alerts to the user's personal physician in case of an important health issue.

In this example, the user may interact with a display (e.g. a touch screen display) or other input device, included in the feedback module 26 within the wearable device 1 in order to set or update information, including location, personal information, medical background, medical data, training processes for activity recognition, or receive health-related notifications. In another example, the user may also specify and receive health-related notifications through the display unit 53 included in the ECU 21.

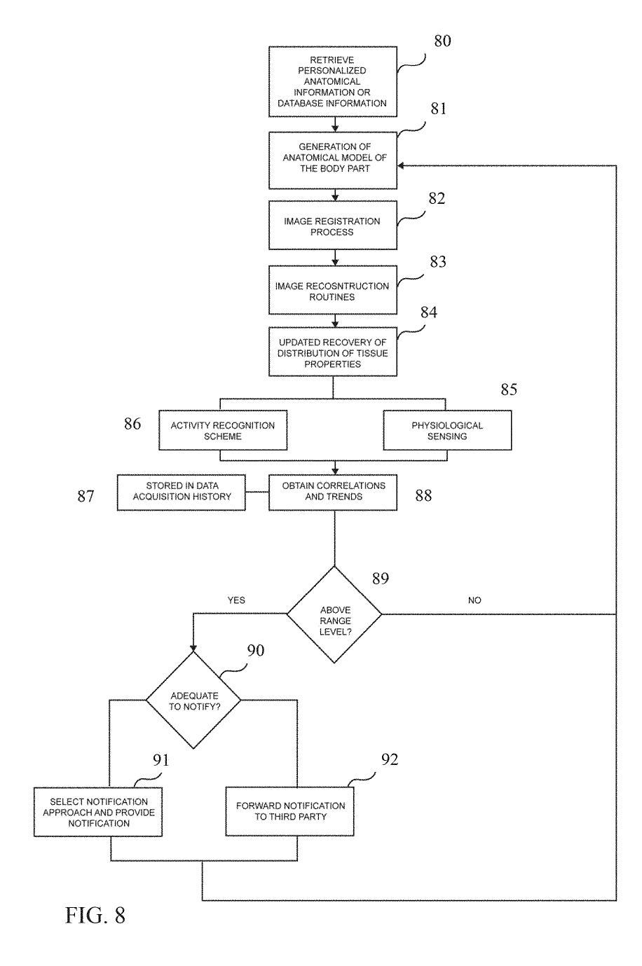

A method for determining and providing health-related notifications to a user will be described with additional reference to FIGS. 8-11. The method may include positioning the wearable device 1 proximate to a body part of the user to retrieve and process anatomical information 80 about the body part, and generate 81 an anatomical model of the body part based upon the anatomical information. The anatomical information may include previously acquired medical image data or other type of data available in health databases or other informatics tools, projects or initiatives/exchange such as those related to age matched healthy controls. The method includes an image registration process 82 for adjustment of the sensing devices for performance control. Generating and communicating such adjustment signals for the sensing devices 2-5 based upon the image registration process 82 may include mapping a position and arrangement of the sensing devices relative to anatomical structures 29 defined in the anatomical model 27 to generate a mapped anatomical model, and generating tissue-related measurements within the mapped anatomical model based upon targeted anatomical structures 29 within the sensitivity path 28 relative to the sensing devices.

The method steps are preferably iterative and include further image reconstruction routines 83 and updated 84 recovery of distribution of tissue properties over time, for example, as illustrated in the chart of FIG. 3. Obtaining measurements from the sensing devices and comparing the measurements with the local energy-related values to determine tomographic data of tissue properties regarding the body part may be included. Such steps provide physiological parameter determination 85 and activity recognition 86.

The method ultimately determines and provides health-related notifications 91, 92 to a user. Physiological parameters 85 determined by the method may be stored 87 and analyzed for further display to the user.

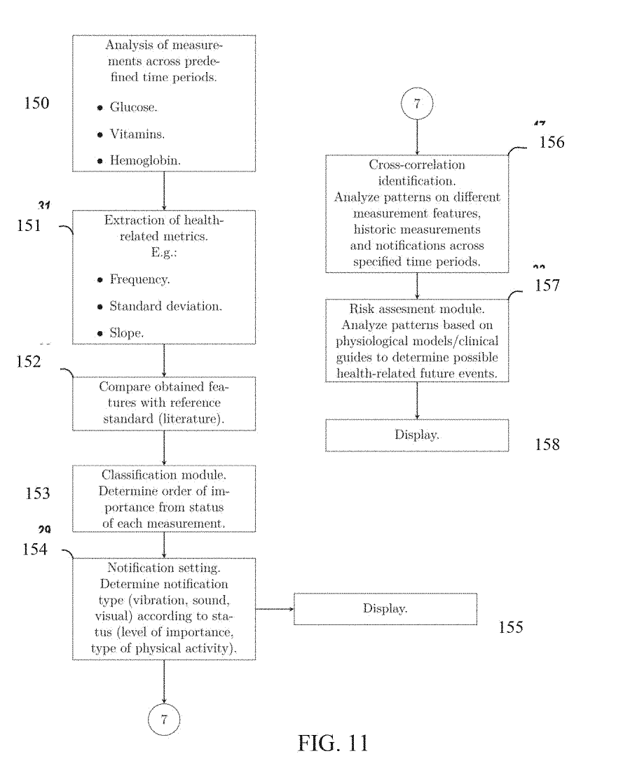

With reference to FIG. 11, the method may include analyzing 150 measurements (e.g. glucose, vitamins, hemoglobin, etc.) across pre-defined time periods, and extracting 151 health-related metrics that may be compared 152 to reference standards in the literature or against past registries from the user in order to determine 153 the relative importance and determine the appropriate health-related notifications 154. The feedback module 26 may then provide the health-related notifications and it may supply 155 haptic, audible and visual notifications (e.g., via a display), depending on the type of notification that it is desired to deliver to the user.

The method may include identifying 88 correlations and trends based upon the health-related metrics to determine and provide the health-related notifications to the user by, for example, evaluating the levels 89, and the determine 90 whether notifications are needed or otherwise appropriate.

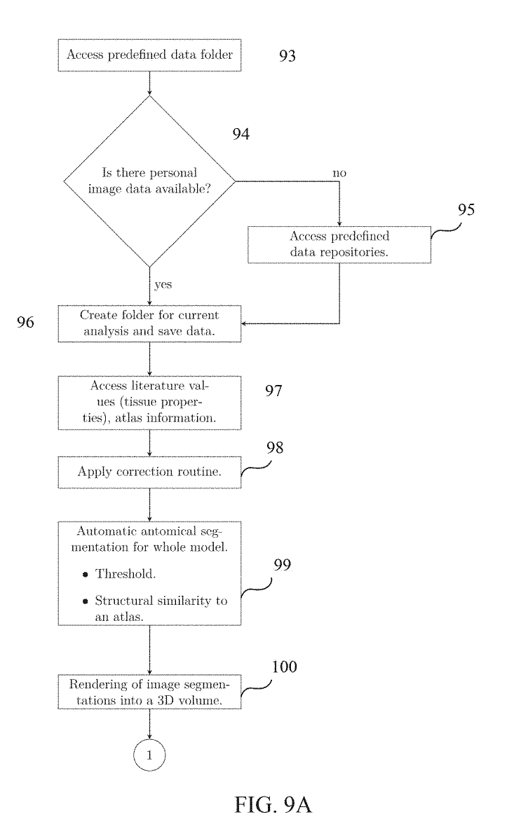

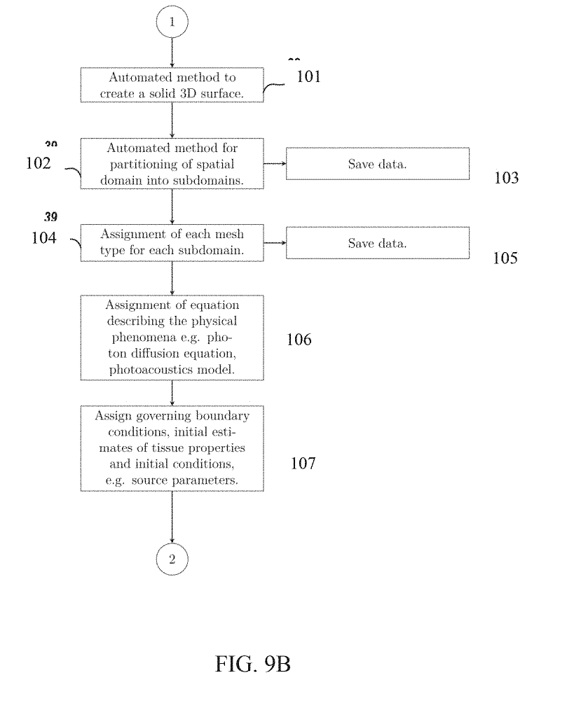

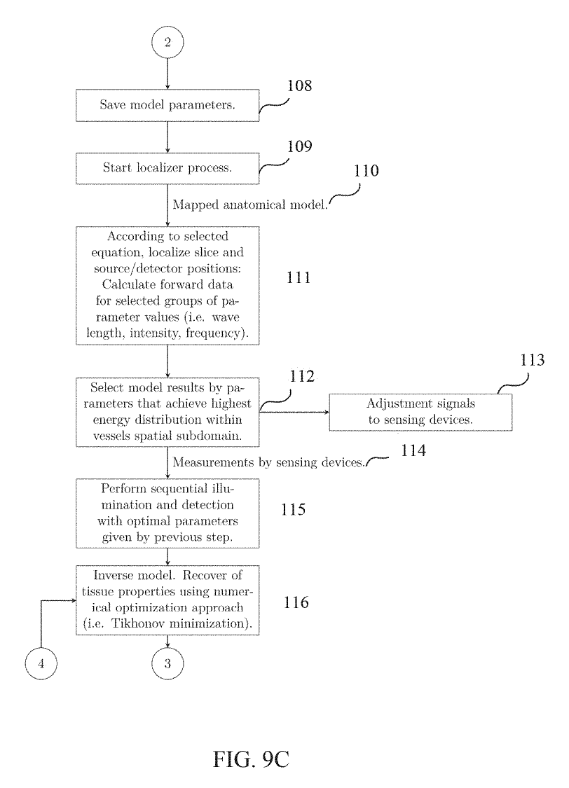

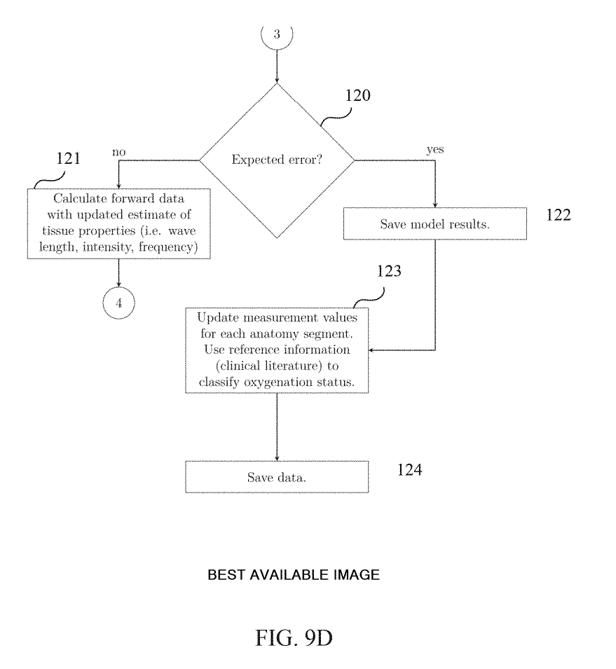

Generating 81 the anatomical model of the body part may be obtained based upon the anatomical information retrieved from a pre-established server, for example, as shown in the flowchart of FIG. 9A. Folders may be accessed 93 to look 94 for personal image data of the user. If there is no personal data, then general pre-defined data may be used 95. At 96 a folder may be created for current analysis and data storage. Also, known values for tissue properties may be accessed 97. Preprocessing steps 98 such as slice timing correction, motion correction and filtering may be used to reduce noise up to a certain threshold level or another desired quality parameter across different anatomical data sets. Automated or semi-automated segmentation methods 99 using anatomical prior information from the patient and/or literature knowledge may be employed in order to generate tissue probability maps for different tissue structures such as arteries, veins, bones, as well as muscles, ligaments, dermal layers, etc. Segmentation results may be then used to generate geometrical shapes related to the various anatomical structures and smoothed to derive 3D-computer-aided (CAD) surfaces 100.

With additional reference to FIG. 9B, segmentation-free approaches may also be considered for utilizing the anatomical-prior image contrast directly on further registration, modeling and image reconstruction steps. 3D CAD surfaces 101 may be then meshed and processed to generate an anatomical model of the body part including details of the various anatomical structures. Further processing, such as, partitioning 102 of spatial domain into subdomains, and assignment 104 of each mesh type for each subdomain may be included, and the data stored 103/105. Equations may be assigned 106 to describe the physical phenomena, as well as the assignment 107 of boundary conditions, initial estimates of tissue properties and initial conditions.

With additional reference to FIG. 9C, the image registration process 82 may include mapping a position and arrangement of the sensing devices relative to anatomical structures defined in the anatomical model to generate a mapped anatomical model 110. After the model parameters are stored 108, the image registration process 82 may include a localizer process 109 in order to map a position or set of positions of the sensing devices relative to predefined anatomical landmarks within the anatomical model, such as the position of the cubital bone, by performing several reference measurements with the sensing devices. The reference measurements may be then compared with the results of multi-physics modeling schemes using the anatomical model, the set of operational parameters of the sensing devices employed in the reference measurements (i.e. wavelength, frequency, intensity, amplitude, modulation, etc.) and reference or updated estimates of the tissue properties (i.e. optical properties, acoustic properties, electrical properties, etc.) for each of the different tissue structures 29 comprised on the anatomical model. Relationships between the model results at each node across the boundary and the measurements performed by each of the detectors may be analyzed in order to determine a mapped anatomical model, where the relative positions of the energy detectors along the boundary and with respect to the underlying anatomical structures within the anatomical model are defined, as shown in FIG. 2A. Resulting positions of the sensing devices relative of the anatomical landmarks on the anatomical model may be employed to compute the anatomical mapped model 110 with details on the relative positions of the sensing devices, based on the known physical spacing and arrangements of the sensing devices in the wearable device 1. Forward data may be calculated 111.

Tissue-related measurements 112 within the mapped anatomical model 110 may be generated by determining local energy-related values for targeted anatomical structures (e.g. veins, arteries) relative to the sensing devices. Multiphysics modeling scenarios may be developed based on a set of different model parameters related to the positions of sensing devices (e.g. wavelength, intensity, frequency). Tissue-related measurements may be then obtained for each scenario by determining local energy-related values for the targeted anatomical structures 29. Model parameters corresponding to the tissue-related measurements that achieve the highest local energy distributions within the targeted anatomical structures are then selected to determine adjustment signals 113 for performance control of the sensing devices 112.

Adjustment signals 113 may be determined via multi-parameter optimization to ensure best combination in terms of wavelength, power intensity, signal-to-noise ratio, or other optimization parameters. etc. Adjustment signals 113 may be defined based on the selected model parameter by generating control signals to the sensing devices. Adjustment signals 113 may be control signals dictating changes on wavelength or amplitude, deactivation or reactivation of specific set of sensors, etc. Also, sequential illumination and detection with optimal parameters may be performed 115, as well as recovery of tissue properties using numerical optimization 116.

Tomographic data of tissue properties 121 regarding the body part may be determined based on the mapped anatomical model and the adjustment signals 113. For example, adjustment signals may be stored in the memory of the processor of the ECU 21, and communicated to the processor 18 of the wearable device 1, which then may communicate the data to the sensing devices for performance control (i.e. adjustment of operational parameters). The ECU 21 may start an image reconstruction routine 83 given by a sequential set of measurements performed by the sensing devices included in the wearable device (e.g. sequential illumination and detection) 115. A model-based inversion routine 116 may be performed by comparing the previously obtained multiphysics model results 42 corresponding to the highest energy distributions to the targeted anatomical structures, with the set of measurements 114 performed by the sensing devices. The inversion routine may consider the measurements located in the nodes of the mesh corresponding to the energy detectors (i.e. boundary). The inversion routine may be repeated until an expected error is obtained 120 (FIG. 9D).

Model results may be saved 122, and tissue properties and physiological parameters of the user that result in the minimal error may be recovered as a result of the inversion routine. Examples of tissue properties include the optical absorption coefficients of main chromophores such as oxyhemoglobin and deoxyhemoglobin. Examples of related physiological parameters 30 include concentration estimates of oxyhemoglobin and deoxyhemoglobin. The concentration of each chromophore may be extracted as absolute or relative measurements.

Physiological parameters 30 may be analyzed over a time period to obtain health-related metrics 31. Examples of health-related metrics relative to each physiological parameter and time period may include frequency, standard deviation, rate of change, etc. Cross-correlations 156 may be obtained by analyzing different physiological parameters and health-related metrics over a time period. Analysis of health-related metrics and correlations may include the use of reference/standard information 152 (e.g. physiological models/clinical guides) and measured historical physiological parameters from the user 156 in order to identify possible health-related risks 157. A notification setting may include criteria for providing notifications to the user and the type of notification to be used (e.g. visual, auditory, visual). A notification setting may be designed based on clinical guides, contextual information pertaining to the type and intensity of the activity being performed by the user, etc.

In an exemplary embodiment of the device, feedback provided by the pressure sensing devices 49 along with signal quality measures may indicate lost skin contact, which may generate adjusting signals to trigger the compressive mechanism 48, as shown in FIG. 2B and FIG. 2C in order to maintain skin contact. The compressive mechanism 48 may include reprogrammable materials that may reversibly change their mechanical, electrical, optical and magnetic properties in response to the adjustment signals delivered by the processor. Reprogrammable materials may include polymeric materials within the field of organic electronics, graphene-based electronic devices, and/or other configurable novel materials.

Modeling and image reconstruction routines along with measurements performed by the sensing devices may be carried out continuously, at a rate determined by the user and/or as indicated by computations of signal quality levels related to signal-to-noise ratio (SNR), detection efficiency and other parameters. Modeling may utilize numerical methods and iterative schemes to derive the image reconstruction. For example, the forward problem may involve numerically solving the photon diffusion approximation to the radiative transport equation over the mapped anatomical model. Both continuous-wave and time-domain schemes may be considered along with sequential or frequency encoded acquisitions. Techniques such as regularization, compressed sensing, etc., may be integrated to account for ill-possedness of the numerical framework due to the diffusive-scheme due to transport in turbid media like soft tissue. Source term parameters may be fed into the model according to the current operational parameters of the energy sources (i.e. intensity, frequency, wavelength, modulation, etc.) included in the wearable device along with device specifications (i.e. size, relative spacing, etc.).

The inverse problem may be implemented using iterative optimization schemes to recover the tissue property values that minimize error between the model results and boundary measurements. Images of tissue parameters at all wavelengths and frequencies may be derived across time and stored in the device memory in order to update initial estimates for subsequent image reconstruction processes.

Post-processing applied to resulting parameter images may include the recovery of deep-temperature distributions, concentration changes of important tissue absorbers such as hemoglobin and deoxy-hemoglobin, water, glucose, cholesterol, conductivity values, vitamin levels, cell-count, flow readings, genetic predisposition markers, etc. According to the method of the embodiments, information conceming relative changes in the spatial distribution of physiological parameters, along with pointwise measurements provided by the sensing devices may be continuously stored for subsequent analysis.

Post-processing schemes may be applied for determination of personalized conditions, considering signal processing algorithms, previously obtained clinical information data or other type of standardized physiological reference data for the physiological parameters considered in the wearable device 1. Post-processing schemes may include a machine-learning based three-part framework that probes the resulting image parameter distributions along with pointwise physiological parameters and physical activity recognition (including hydration levels, caloric intake, cardiac frequency, stress-related measures, posture, voice pitch, equilibrium and balance, activity levels, type of activity performed, etc.) in order to find correlation and trends within data sets and predict possible future health-related events. Training, feature extraction and signal classification steps may then be applied to determine health-related notifications. Inclusion of portable setups such as Optical Coherence Tomography (OCT), Ultrasound, etc. may be devised in the device to enable determination of changes in the anatomy of the user, including different tissue structures such as subdermal thickness, bone dimensions, etc. Updated anatomical model may be then considered in the modeling and image reconstruction steps.

In an exemplary embodiment of the device, a notification system may be modulated to determine a suitable notification setting including visual, vibratory, acoustic or other type of feedback mechanism to the user or a third party via wireless communication. Assessment of stress-related conditions may also support the modulation of the notification system by correlating salivary measurements with voice-pitch signals or other stress-related measurements provided by the wearable device 1.

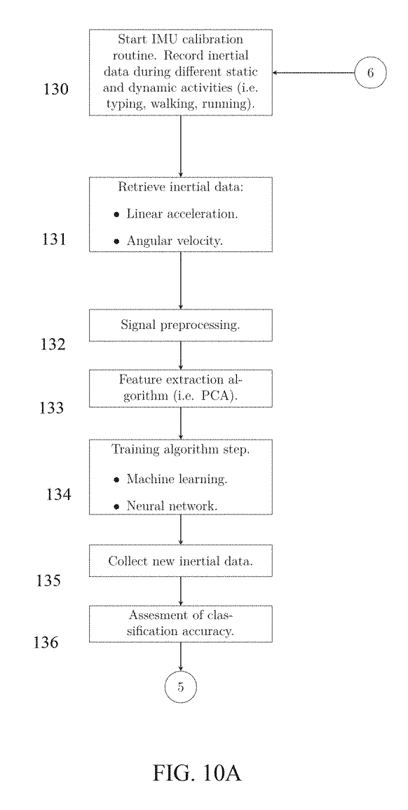

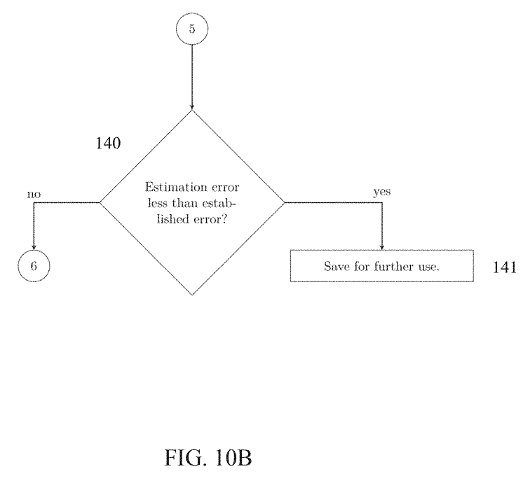

Referring additionally to FIG. 10A, simultaneous determination of the type of activity performed by the user may be achieved using machine-learning approaches including calibrations for the training process. Therefore, physiological assessment combined with simultaneous activity recognition steps may be used to determine whether a particular scenario is convenient and safe for displaying health-related notifications.

Calibration of the IMU may including recording different values 130, retrieving inertial data 131, processing 132, feature extraction 133, training 134 such as machine leaming and/or neural network approaches, collection of new data 135 and the assessment 135 of activity classification accuracy. If the estimated error 140 is below a threshold then the assessment is saved 141 for further use by the approach. If the estimated error is above the threshold then the calibration is begun again.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

That which is claimed:

1. A wearable device configured for attachment to a body part of a user for sensing, feedback and adjusting features, the wearable device comprising:
a flexible housing configured to be attached to the body part of the user;
an array of sensing devices carried by the housing and configured to be positioned proximate the body part to determine respective physiological characteristics of the user;
a processor configured to receive and process information regarding the physiological characteristics from the array of sensing devices;
a memory associated with the processor and configured to store the processed information;
a communication module associated with the processor and configured to communicate data to/from an external control unit (ECU); and
at least one power source configured to provide power to the array of sensing devices and the processor;
wherein the processor is configured to receive adjustment signals from the ECU and adjust the sensing devices for performance control according to an image registration process that includes
mapping a position and arrangement of the sensing devices relative to anatomical structures defined in an anatomical model to generate a mapped anatomical model, and
generating tissue-related measurements within the mapped anatomical model based upon targeted anatomical structures within a sensitivity path relative to the sensing devices.

2. The wearable device according to claim 1, wherein the flexible housing comprises at least one of an elastic band, a breathable, stretchable, conformable band, and a shape-memory polymer band.

3. The wearable device according to claim 1, wherein the flexible housing further comprises a compression mechanism controlled by the processor to adjust the fit and position of the sensing devices.

4. The wearable device according to claim 1, wherein the array of sensing devices is configured to determine physiological characteristics using at least one of an electroencephalogram (EEG), electrooculogram (EOG), electromiogram (EMG), electrocardiogram, (ECG), optical spectroscopy and tomography, optical photoacoustic spectroscopy and tomography, thermoacoustic spectroscopy and tomography, bioimpedance, bioimpedance tomography, acoustic sensing, enzyme-based and enzyme-free electrochemical sensing, temperature sensing, inertial mass unit measurements, and combinations thereof.

5. The wearable device according to claim 1, wherein the array of sensing devices comprises at least one of optical sensor arrays, acoustic sensor arrays, photoacoustic sensor arrays, bioimpedance sensor arrays, electrochemical biosensors, and inertial measurement units (IMU).

6. The wearable device according to claim 1, wherein the array of sensing devices includes at least one of lasers, solid state light sources, light emitting diodes (LEDs), electro-acoustical transmitters, piezoelectric transducers, voltage electrodes, current electrodes, alternating voltage generators, voltage-to-current converters, colorimeters, miniaturized spectrometer systems, miniaturized charge-coupled devices (CCDs), photodiodes, avalanche photo-diodes (APD), phototransistors, photo-multiplying tubes, hybrid photodetector systems, optical fiber-based sensor systems, and micro resonator systems.

7. The wearable device according to claim 1, wherein the array of sensing devices are arranged in one or more rows within the flexible housing and include at least one of point measurements and tomographic measurements to provide at least one of transverse and axial image information as the physiological characteristics of the user.

8. The wearable device according to claim 1, wherein the array of sensing devices comprise reprogrammable materials configured to controllably change at least one of mechanical, electrical, optical and magnetic properties in response to the adjustment signals that include at least one of electronic signals, magnetic signals, and electromagnetic signals.

9. The wearable device according to claim 1, wherein the communication module associated with the processor and configured to communicate data to/from the ECU comprises at least one of a wireless communication module and a wired communication module.

10. The wearable device according to claim 1, further comprising a feedback module associated with the processor and configured to provide health-related notifications to the user using at least one of audible, visual and haptic notifications.

11. A method for determining and providing health-related notifications to a user, the method comprising:
providing a wearable device, including an array of sensing devices, to be positioned proximate to a body part of the user;
retrieving and processing anatomical information about the body part;
generating an anatomical model of the body part based upon the anatomical information;
generating and communicating adjustment signals for the sensing devices based upon an image registration process that includes
mapping a position and arrangement of the sensing devices relative to anatomical structures defined in the anatomical model to generate a mapped anatomical model, and
generating tissue-related measurements within the mapped anatomical model based upon targeted anatomical structures within a sensitivity path relative to the sensing devices;
determining local energy-related values within the anatomical model;
obtaining measurements from the sensing devices and comparing the measurements with the local energy-related values to determine tomographic data of tissue properties regarding the body part;
determining physiological parameters of the user based upon the tomographic data;
analyzing the physiological parameters over a time period to obtain health-related metrics; and
identifying correlations and trends based upon the health-related metrics to determine and provide the health-related notifications to the user.

12. The method according to claim 11, wherein the wearable device includes an elastic band carrying the array of sensing devices to be positioned adjacent the skin of the body part.

13. The method according to claim 12, wherein the elastic band further comprises a compression mechanism controlled to adjust the fit and position of the sensing devices.

14. The method according to claim 11, wherein obtaining measurements from the sensing devices includes using at least one of an electroencephalogram (EEG), electrooculogram (EOG), electromiogram (EMG), electrocardiogram, (ECG), optical spectroscopy and tomography, optical photoacoustic spectroscopy and tomography, thermoacoustic spectroscopy and tomography, bioimpedance, bioimpedance tomography, acoustic sensing, enzyme-based and enzyme-free electrochemical sensing, temperature sensing, inertial mass unit measurements, and combinations thereof.

15. The method according to claim 11, wherein the array of sensing devices comprises at least one of optical sensor arrays, acoustic sensor arrays, photoacoustic sensor arrays, bioimpedance sensor arrays, electrochemical biosensors, inertial measurement units (IMU), lasers, solid state light sources, light emitting diodes (LEDs), electro-acoustical transmitters, piezoelectric transducers, voltage electrodes, current electrodes, alternating voltage generators, voltage-to-current converters, colorimeters, miniaturized spectrometer systems, miniaturized charge-coupled devices (CCDs), photodiodes, avalanche photo-diodes (APD), phototransistors, photo-multiplying tubes, hybrid photodetector systems, optical fiber-based sensor systems, and micro resonator systems.

16. The method according to claim 11, wherein the array of sensing devices are arranged in one or more rows within the wearable device and include at least one of point measurements and tomographic measurements to provide at least one of transverse and axial image information as the tomographic data of the user.

17. The method according to claim 11, wherein the array of sensing devices comprise reprogrammable materials; and further comprising controllably changing at least one of mechanical, electrical, optical and magnetic properties of the sensing device in response to the adjustment signals that include at least one of electronic signals, magnetic signals, and electromagnetic signals.

18. The method according to claim 11, wherein retrieving and processing anatomical information about the body part includes communicating data from the wearable device to an external control unit using at least one of a wireless communication module and a wired communication module of the wearable device.

19. The method according to claim 18, wherein retrieving and processing anatomical information about the body part also includes accessing stored reference anatomical information about the body part including at least one of previously determined physiological parameters of the user and general physiological parameters of the body part.

20. The method according to claim 11, wherein determining local energy-related values within the anatomical model comprises determining at least one of optical properties, acoustic properties, and electrical properties for different tissue structures within the anatomical model using operational parameters of the sensing devices including at least one of wavelength, frequency, intensity, amplitude, and modulation.

21. The method according to claim 11, wherein analyzing the physiological parameters to obtain health-related metrics includes obtaining at least one of deep-temperature distributions, concentration changes of hemoglobin and deoxy-hemoglobin, water, glucose, cholesterol, conductivity values, vitamin levels, cell-count, flow readings, and genetic predisposition markers.

22. The method according to claim 21, wherein obtaining health-related metrics further includes obtaining at least one of hydration levels, caloric intake, cardiac frequency, stress-related measures, posture, voice pitch, equilibrium and balance, activity levels, and type of activity performed of the user.

23. The method according to claim 11, wherein identifying correlations and trends based upon the health-related metrics to determine and provide notifications to the user further includes identifying cross-correlations and trends between health-related metrics.

24. The method according to claim 11, wherein providing the health-related notifications to the user includes using at least one of audible, visual, haptic, and electronic notifications.
